SEQUENCE LISTING (2 sequences)

SEQ ID NO: 1 - DNA, 649 bp, Glycine tomentella
ctacactcttcatgggtgagatctctggagcactgcaccaagccatacctcttctcagtggaactcaaattgaagccatccatatagtacaactgattggtctcattagttgcttttctgatcaaacttctcataaaaaatcctgccttttctgaatctctgccatgttggaaatgtttttggctccaacatcatgccatgttgagaatgttgtaacattcccaaagaagctttcattagagtaccttatgacgcagaaatcataccatatgatagcagaaactctattgaggcagtgctgaagtacttgtctggaagcagtagatacacagaattgacaaaagtatccaaacacatcaccacggcaatcatagaggccatacacagcactgtagttgttttttcctatcgttttgtggttataacctttgcttgtggctgcatctgtggatagccatgagagtatgctgttaaggttggtttggtatgcatcgctgagaggtttttgggtggtgttatggcagtcatctcccatgtagtttggtgattgtgcctttgtatcaagtggtttgatgcttagcaatagcaggacagcaaaggaatatactgatttcacactgagtagtgttgcatccattgagcactgtct

SEQ ID NO: 2 - DNA, 596 bp, Glycine syndetika
aataagtacaccacctgaccaccattttgcataaacaccttattttgcacaaactacaacagaaatataagaaaacacaacccagatgggaaagaaaagtacaagatttgtttgctttaccaacatcagatcagctactacattatccactttagagttctgtacctttgccctctatagtgttcataaacaaagccaaactgaggaacaagcatgtaaagcaagtcatcaacacgctgccaatacaaaagcccccacctctccaagagcataaggggaaggataatgattccaaagccaaaaatgaatcccagctccacacttaggaaattccagtcaattgatccatgtatttcatatagtgttggaagggaatgtcccactctatcgttggtgcaatcttttaagggtggcccacataatccttcattgccttcaaaataacttgcatcaaatgtttgcatttgagcacctgttgggatttgccccctcaactgattaaatgagagattcaagtatgctagaaaatttaaactagcaagctcaagagggatttttccgctcaaattgttatttgacatgtccaaggattcaag

(19) United States
(12) Patent Application Publication
(10) Pub. No.: US 2019/0390220 A1
(43) Pub. Date: Dec. 26, 2019

(54) Compositions and Methods of Increasing Aphid Resistance in Transgenic Plants

(71) Applicants: Georg Jander, Ithaca, NY (US); Jing Wei, Ithaca, NY (US)

(72) Inventors: Georg Jander, Ithaca, NY (US); Jing Wei, Ithaca, NY (US)

(73) Assignee: Boyce Thompson Institute, Ithaca, NY (US)

(21) Appl. No.: 16/269,199

(22) Filed: Feb. 6, 2019

Related U.S. Application Data
(60) Provisional application No. 62/629,261, filed on Feb. 12, 2018.

(51) Int. Cl. C12N 15/82; G01N 33/50

(52) U.S. Cl. CPC C12N 15/8286; G01N 33/5097

(57) ABSTRACT

The present invention relates to isolated resistance genes encoding polypeptides that confers resistance to aphid infestation, and methods of transforming plant and cells with the disclosed resistance genes. The invention also relates to transgenic plants and cells having resistance to aphids, particularly Aphis glycines.

### CROSS-REFERENCE TO RELATED APPLICATIONS

This application claims the benefit of priority of U.S. Provisional Application Ser. No. 62/629,261, filed Feb. 12, 2018, the contents of which are herein incorporated by reference in their entirety.

### FIELD OF THE INVENTION

This invention relates to the fields of transgenic plants and pest resistance in higher plants. More specifically, the invention provides compositions and methods for enhancing aphid resistance in transgenic soybean and other plants.

### BACKGROUND OF THE INVENTION

Several publications and patent documents are cited throughout the specification in order to describe the state of the art to which this invention pertains. Each of these citations is incorporated herein by reference as though set forth in full.

The legume genus Glycine, which contains the annual subgenus Soja, as well as the perennial Glycine species, experienced a polyploidy event 5-13 million years ago (13). The subgenus Soja includes the cultivated soybean, G. max, and its wild progenitor, G. soja, which is native to southeast Asia. The perennial subgenus Glycine is native to Australia and neighboring Papua New Guinea (14). A second round of genome duplication occurred in the subgenus Glycine around 500,000 years ago (15) through the formation of allotetraploids (2n=80) from various combinations of extant diploid progenitor species (16), several of which have colonized islands of the Pacific Ocean (17). One of the natural allotetraploids, Glycine dolichocarpa (T2, 2n=80), resulted from crosses between two diploid species G. tomentella (D3, 2n=40) and Glycine syndetika (formerly referred to as G. tomentella D4, 2n=40) within the last 0.5 million years (FIG. 2a) (14). Analysis of plastid genomes indicates that G. dolichocarpa arose independently on more than one occasion with either G. tomentella D3 or G. syndetika as the female parent. Relative to its diploid progenitors, G. dolichocarpa has been reported to have enhanced photosynthesis, photoprotection and nodulation (18-21).

Aphids are common pests of soybean, causing damage both through the direct effects of feeding and by vectoring debilitating plant viruses. The soybean aphid, Aphis glycines Matsumura, is a significant insect pest of soybean in the north-central region of the United States, causing substantial yield loss (22). As increased aphid resistance would enhance soybean productivity, a number of studies have attempted to attain this by using diverse G. max germplasms (23, 24). Unfortunately, the currently available soybean aphid (SBA) resistance genes identified from the G. max and its wild relative G. soja, including 5 Rag (resistance to Aphis glycines) genes, have been overcome by the occurrence of new soybean aphid biotypes (24-26). The pea aphid (PA), Acyrthosiphon pisum Harris, is another serious pest for many legume crops and, like the soybean aphid, is prone to forming sympatric populations showing differential preferences and fitness on specific host plants (27-29). Resistance to pea aphids has been identified in some legumes (30-33), but to date no resistance has been identified in soybean. Continued screening of soybean germplasm is needed to identify new aphid resistance alleles.

### SUMMARY OF THE INVENTION

In accordance with the present invention, isolated polynucleotides of SEQ ID NOS: 1 and 2 or sequences having at least 95% identity to SEQ ID NO: 1 or SEQ ID NO: 2; optionally comprising operably linked promoter sequences operable in a higher plant are provided. Also provided are expression cassettes encoding one or both of SEQ ID NOS: 1 and/or 2. Such expression cassettes are conveniently placed with in a recombinant vector suitable for use in plants. In certain embodiments, the vector is selected from the group consisting of a plasmid, a viral vector, and an agrobacteria vector.

In one embodiment, a transgenic Glycine max plant cell comprising the polynucleotides described above is provided. Also encompassed within the present invention is a method for producing transgenic plant cells and transgenic soy bean plants resistant to aphid infestation. An exemplary method comprises contacting a Glycine max plant cell with a plant transformation vector, and regenerating a transgenic plant therefrom.

The present invention also provides a method for altering expression levels of SEQ ID NOS: 1 and/or 2 in plant cells in a method for screening agents which modulate aphid infestation pathways. In one approach, siRNA molecules are employed to reduce expression of SEQ ID NOS: 1 and 2. In other embodiments, these sequences are overexpressed to increase expression levels of the proteins encoded thereby.

### BRIEF DESCRIPTION OF THE DRAWINGS

FIG. 1: Reproduction of soybean aphids and pea aphids on diploids (G. tomentella D3 and G. syndetika D4) and allotetraploid (G. dolichocarpa T2) wild perennial soybean species, and overview of soybean transcriptomic responses to aphid feeding. (a) Schematic representation of the experimental designs for aphid bioassays and assays of gene expression by Illumina sequencing. The three-week-old plants were caged, three newborn aphids were added to each plant, the number of aphid was counted after one week and leaf tissue was harvested for metabolites analysis. Data are mean+/−s.e. of N=60 (6 individuals each of 10 natural isolates of each soybean species). Different letters indicate significant differences, P<0.05, ANOVA followed by Tukey's HSD test. (b-c) Pea aphid (b) and soybean aphid (c) performance on two soybean diploids D3 and D4, as well as their allotetraploid T2.

FIG. 2: The Glycine study system. (a) Phylogenetic relationship between soybean, G. max and members of subgenus Glycine included in this study, G. tomentella D3, G. syndetika D4, and G. dolichocarpa T2. (b) Australian and Papua New Guinea locations for accessions used in this study. One accession of G. dolichocarpa from Taiwan was also used, but is not shown here.

FIG. 3: Overview of soybean transcriptomic responses to pea aphid (PA) and soybean aphid (SBA) feeding. (a) Schematic representation of the experimental designs for assays of gene expression by Illumina sequencing. At staggered intervals, 15 aphid adults were added to each plant, leaf tissue was harvested after 8 and 48 h of aphid infestation. (b, c) The number of transcripts that were significantly (P<0.05 FDR adjusted, and fold change greater than 2 or less than 0.5.) up- or down-regulated at 8 h (b) and 48 h (c) after initiation of aphid feeding. (d, e) The number of mass features that were significantly (P<0.05, FDR adjusted, and fold change greater than 2 or less than 0.5) increased or decreased at 8 h (d) and 48 (e) after initiation of aphid feeding. (f, g) Heatmaps of enriched KEGG pathways at 8 h (f) or 48 h (g) after initiation of pea aphid or soybean aphid feeding.

FIG. 4: Boxplot of aphid performance on individual accession of the three soybean species. Data are mean+/−s.d. of N=6.

FIG. 5: Plant flavonoid metabolism. (a) Schematic diagram of the flavonoid biosynthesis pathway. The dashed arrow represents possible enzymatic steps. Genes colored in red were measured. (b, c) Heatmap showing expression values for genes indicated in panel a after pea aphid (PA, b) or soybean aphid (SBA, c) feeding for 8 h or 48 h, respectively. Red boxes indicate genes that are significantly upregulated in flavone and isoflavone biosynthesis by PA and SBA, respectively (d, e) Relative expression levels of isoflavone synthase (d) and flavone synthase (e) after aphid feeding. (f, g) Isoflavone (f) and flavone (g) abundance after aphid infestation. Values are mean+/−s.e. of N=3, *P<0.05, ANOVA followed by Dunnett test. PAL=phenylalanine ammonia-lyase, 4CL=4-Cinnamoyl CoA, IFS=isoflavone synthase, IF4′OMT=isoflavone 4′-O-methyltransferase, IF7OMT=isoflavone-7-O-methyltransferase, FLS=flavone synthase, F8OMT=flavonoid 8-O-methyltransferase, F3OMT=flavonol 3-O-methyltransferase, F3′5′MT=flavonoid 3′,5′-methyltransferase.

FIG. 6: Principal component analysis of the transcriptome (a) and metabolome (b) profiles of soybean plants infested by aphids at 0, 8 and 48 h. PA, pea aphid, SBA, soybean aphid, D3, G. tomentella, D4, G. syndetika, and T2, G. dolichocarpa.

FIG. 7: Effects of flavonoids on pea aphid (PA) and soybean aphid (SBA) feeding. (a, b) Number of surviving individuals over time (2-6 days) for pea aphids (a) and soybean aphids (b) exposed to artificial diet containing isoflavones or flavones in oral feeding assays. Data are mean+/−s.e. of N=3 for each flavonoid. *P<0.05, **P<0.01, ***P<0.001, ANOVA followed by Dunnett test. (c, d) Relationships between aphid performance and isoflavones (c) and flavones (d) in leaves collected as shown in FIG. 1a. For each soybean species, seven accessions were chosen for measuring the amount of flavonoids, for each accession, two plants were conducted.

FIG. 8: Overview of the transcriptome assembly. (a) Summary of the de novo transcriptome assembly for D3, D4 and T2. (b) Transcriptomic similarity between mock and aphid infested samples (8 h and 48 h after infestation) in the three genotypes. The color gradients indicate the Pearson correlation coefficients among samples. PA=pea aphid, SBA=soybean aphid, D3=G. tomentella, D4=G. syndetika, and T2=G. dolichocarpa.

FIG. 9: Flavonoids modulate soybean defense against aphids. (a, b) Relative expression level of isoflavone synthase (a) and flavone synthase (b) in control, silenced and overexpressed plants. (c, d) Isoflavones (c) and flavones (d) abundance in control, silenced and overexpressed plants after aphid infestation. (e, f) Pea aphid (e) and soybean aphid (f) performance on control, silenced and overexpressed plants. Data are mean+/−s.e. of N=5. Different letters indicate significant differences, P<0.05, ANOVA followed by Tukey's HSD test.

FIG. 10: Predicted R genes regulate soybean defense against aphids. (a) Classification of predicted genes based on the presence of specific domains. CNL-includes those with at least a coiled-coil domain, a nucleotide binding site and a leucine-rich repeat; RLP-receptor-like protein, with an extracellular leucine-rich repeat; TNL-includes those with a Toll-interleukin receptor-like domain, a nucleotide binding site and a leucine-rich repeat; RLK-contains those with a kinase domain, and an extracellular leucine-rich repeat; Others-other genes which have been described as conferring resistance through different molecular mechanisms. (b) Summary of the predicted R genes identified in D3, D4 and T2. (c-e) Relative expression level of predicted cysteine-rich receptor-like protein kinase 42 (CRK42) (c), putative cysteine-rich receptor-like protein kinase 20 (CRK20) (d) and putative lectin S-receptor-like serine/threonine-protein kinase (LRK) (e) after aphid infestation. (f, h, i) Relative expression levels of CRK42 (f), CRK20 (h) and LRK (i) in control, silenced, and overexpressed plants. (g, I & k) Pea aphid (g) and soybean aphid (I & k) performance on control, silenced, and overexpressed plants.

### DETAILED DESCRIPTION OF THE INVENTION

Here, we report that the allotetraploid perennial soybean Glycine dolichocarpa is resistant to both Aphis glycines (soybean aphid) and Acyrthosiphon pisum (pea aphid), whereas the diploid progenitors, Glycine tomentella D3 and Glycine syndetika, show divergent resistance to the two aphid species. Using transcriptomic and metabolomic approaches to compare responses of the three perennial soybean species to aphid infestation, we found that they vary in their responses to A. glycines and A. pisum. Perennial soybeans resistant to A. pisum accumulate more isoflavones in response to aphid attack, whereas those resistant to A. glycines accumulate more flavones. This is recapitulated in artificial diet assays, where isoflavones have a greater negative effect on A. pisum and flavones have a greater negative effect on A. glycines. Correlative analysis of gene expression and aphid resistance in the three perennial soybean species identified likely resistance (R) genes. The functions of two of these, the GdCRK20 and GdCRK42 leucine rich repeat receptor kinases, were confirmed by showing that expression silencing and overexpression, respectively, have a significant effect on aphid reproduction. Together, the observation of additive effects of flavonoids and R genes in aphid resistance support the hypothesis that allotetraploidy in perennial soybeans provides an evolutionary advantage through the combination of two plant defense systems.

### Definitions

The phrase “R gene function” is used herein to refer to any R gene activity, including without limitation expression levels of R gene, R gene protein activity, and/or modulation of resistance to pests such as aphids. An “R gene homolog” is any protein or DNA encoding the same which has similar structural properties (such as sequence identity and folding) to the R genes encoded by SEQ ID NOS: 1 and 2.

The term “pathogen-inoculated” refers to the inoculation of a plant with a pathogen or pest.

The phrase “disease defense response” refers to a change in metabolism, biosynthetic activity or gene expression that enhances a plant's ability to suppress the replication and spread of a microbial pathogen (i.e., to resist the microbial pathogen). Examples of plant disease defense responses include, but are not limited to, production of low molecular weight compounds with antimicrobial activity (referred to as phytoalexins) and induction of expression of defense (or defense-related) genes, whose products include, for example, peroxidases, cell wall proteins, proteinase inhibitors, hydrolytic enzymes, pathogenesis-related (PR) proteins and phytoalexin biosynthetic enzymes, such as phenylalanine ammonia lyase and chalcone synthase (Dempsey and Klessig, 1995; Dempsey et al., 1999). Such defense responses appear to be induced in plants by several signal transduction pathways involving secondary defense signaling molecules produced in plants.

A “transgenic plant” refers to a plant whose genome has been altered by the introduction of at least one heterologous nucleic acid molecule.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90 95% by weight of the given compound. Purity is measured by methods appropriate for the given compound (e.g. chromatographic methods, agarose or polyacrylamide gel electrophoresis, HPLC analysis, and the like).

A “replicon” is any genetic element, for example, a plasmid, cosmid, bacmid, phage or virus, that is capable of replication largely under its own control. A replicon may be either RNA or DNA and may be single or double stranded.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention is chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting pAID mRNA, for example, may be between 15-35 nucleotides in length, and more typically about 21 nucleotides in length.

A “vector” is any vehicle to which another genetic sequence or element (either DNA or RNA) may be attached so as to bring about the replication of the attached sequence or element.

An “expression cassette” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15 to 25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “promoter region” refers to the 5′ regulatory regions of a gene (e.g., CaMV 35S promoters and/or tetracycline repressor/operator gene promoters, opine promoter, rice actin promoter, and/or plant ubiquitin promoters.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion, biolistic delivery, and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “DNA construct” refers to a genetic sequence used to transform plants and generate progeny transgenic plants. These constructs may be administered to plants in a viral or plasmid vector. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

The phrase “double-stranded RNA mediated gene silencing” refers to a process whereby target gene expression is suppressed in a plant cell via the introduction of nucleic acid constructs encoding molecules which form double-stranded RNA structures with target gene encoding mRNA which are then degraded.

The term “co-suppression” refers to a process whereby expression of a gene, which has been transformed into a cell or plant (transgene), causes silencing of the expression of endogenous genes that share sequence identity with the transgene. Silencing of the transgene also occurs.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

“Mature protein” or “mature polypeptide” shall mean a polypeptide possessing the sequence of the polypeptide after any processing events that normally occur to the polypeptide during the course of its genesis, such as proteolytic processing from a polyprotein precursor.

A low molecular weight “peptide analog” shall mean a natural or mutant (mutated) analog of a protein, comprising a linear or discontinuous series of fragments of that protein and which may have one or more amino acids replaced with other amino acids and which has altered, enhanced or diminished biological activity when compared with the parent or nonmutated protein.

The present invention also includes active portions, fragments, derivatives and functional or non-functional mimetics of R gene-related polypeptides, or proteins of the invention. An “active portion” of such a polypeptide means a peptide that is less than the full length polypeptide, but which retains measurable biological activity.

A “fragment” or “portion” of an R gene-related polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. Fragments of the R gene-related polypeptide sequence, antigenic determinants, or epitopes are useful for eliciting immune responses to a portion of the R gene-related protein amino acid sequence for the effective production of immunospecific anti-R protein antibodies.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

A “clone” or “clonal cell population” is a population of cells derived from a single cell or common ancestor by mitosis.

A “cell line” is a clone of a primary cell or cell population that is capable of stable growth in vitro for many generations.

The materials and methods set forth below are provided to facilitate the practice of the present invention.

### Plant Growth and Insect Rearing

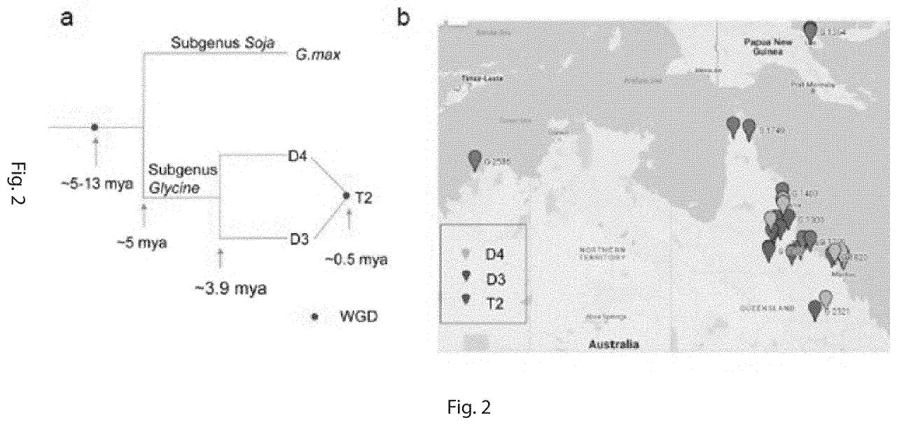

Seeds of 10 accessions each of three Glycine subgenus allotetraploid G. dolichocarpa (T2) and its diploid progenitors G. tomentella (D3), G. syndetika (D4), originally collected from Australia, Papua New Guinea and Taiwan (FIG. 2B) (63), were propagated in a growth chamber and a greenhouse. Seeds were planted in Sunshine MVP RSi Soil (Sun Gro Horticulture, Bellevue, Wash., USA) in Conviron growth chambers under a 16:8 h day:night cycle at 23° C. with a 60% relative humidity for ˜3 weeks. Older plants were moved to a greenhouse with natural sunlight.

A pea aphid colony (A. pisum strain CWR09/18, kindly supplied by Angela Douglas, Cornell University) was reared on faba bean plants (Viciafaba, var. Windsor; Johnny's Selected Seeds, Winslow, Me., USA) which were grown in Metromix 200 (Scotts, Marysville, Ohio, USA) in a growth chamber at the same condition with soybeans in insect rearing room at 25° C. with a 12:12 h day:night cycle (64). A soybean aphid colony (A. glycines), kindly supplied by Gustavo Macintosh at Iowa State University, was reared on soybean plants (G. max var. William 82) that were grown in same soil and conditions as described for the pea aphid colony.

### Insect Bioassays

For initial aphid performance assays, ten accessions of each species were used, and at least 6 plants were tested for each accession. Three newborn aphids were confined on 3-week-old seedlings using plastic cup cages. After 7 days, the number of aphid nymphs was counted. Plants with less than three adult aphids were excluded from the data analysis.

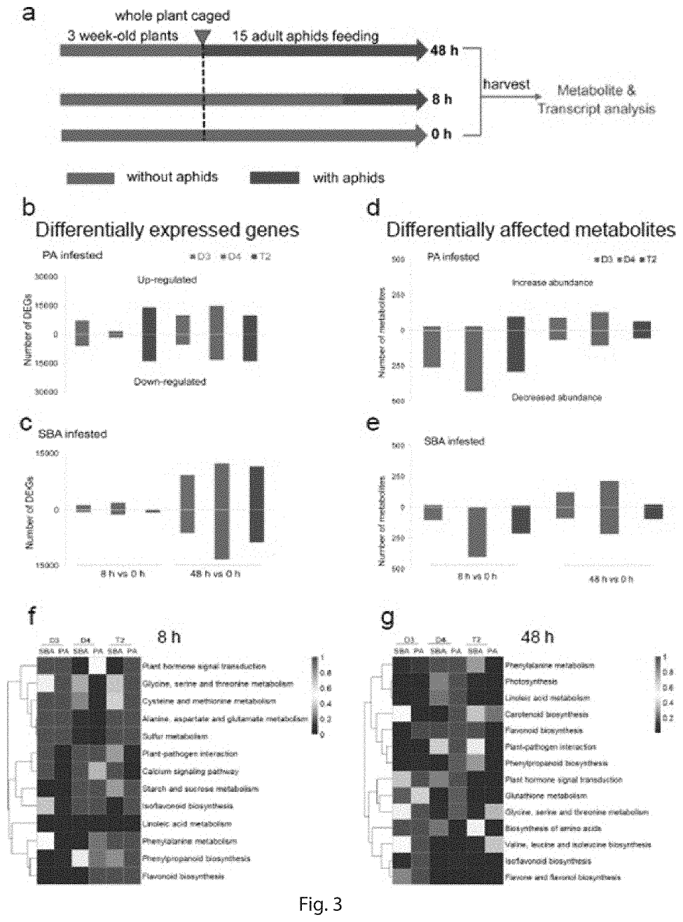

For the time-course bioassay, 15 adult aphids were confined on leaves of 3-week-old seedlings for 8 and 48 h using cup cages. All plants were caged at the start of the experiment and the addition of aphids was staggered (FIG. 3a), so that all plant tissue for gene expression and metabolite assays was harvested at the same time (48 h after the start of the experiment). Control plants were also caged without aphids for 48 h.

For flavonoid in vitro feeding bioassays, the effects of three isoflavones, daidzein, formononetin and prunetin, as well as two flavones, apigenin and kaempferol on A. glycines and A. pisum adults were tested by rearing 30 insects on an artificial diet (65) in membrane feeding tubes. Adults were fed an artificial diet containing one of the following treatments, 0.01% apigenin, 0.2% daidzein, 0.01% prunetin, 0.04% formononetin or 0.02% kaempferol for 6 days in an insect rearing room (25° C. with a 12:12 h day:night cycle). Since dimethyl sulfoxide (DMSO) was used to dissolve these compounds, an artificial diet containing 4 μl/ml DMSO was used for controls. The compound concentrations for diet experiments were based on their measured concentrations in soybean leaves. After 6 days of feeding, the number of surviving aphids was counted.

### Metabolite Assays

For non-targeted metabolomes, soybean leaves infested by SBA or PA from 6-10 accessions of D3, D4 and T2 were collected from the time-course assay (FIG. 3a) and stored in −80° C. Tissue samples were weighed and all data were normalized relative to the tissue fresh weight. Leaf powder (˜100 mg) was extracted in 80% methanol and 20% (v/v) water, samples were centrifuged three times at 15,000 g for 10 min, and the clear supernatants were collected. Two microliters of the supernatant were injected into a Q-Exactive hybrid quadrupole-orbitrap mass spectrometer (Thermo Scientific). Separation of metabolites was achieved on a Kinetex® 1.7 μm EVO C18 100 Å, 150×2.1 mm LC Column (Phenomenex) using the following linear gradient: mobile phase B (95% acetonitrile) increased from 0 to 100% over 14 min, held at 100% for 5 min, and returned to 100% mobile phase C (0.1% acetic acid in water) for equilibration for 1 min. The flow rate of the mobile phases was 0.5 ml/min, and the column temperature and the autosampler temperature were maintained at 40° C. and 10° C., respectively. The analytical mode was in the negative ionization mode. The data matrix was processed using the XCMS (http://bioconductor.org/packages/xcms/) and CAMERA (http://bioconductor.org/packages/CAMERA/) software packages in R. Processed negative ionization data set was transferred to Microsoft Excel for further analysis.

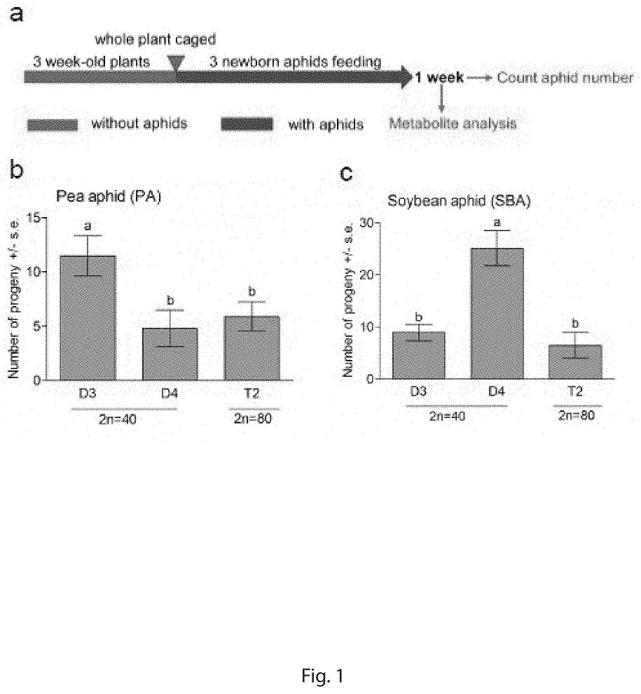

For targeted soybean metabolite assays, leaves infested by SBA and PA from D3_3, D4_7 and T2_10 were collected at 0, 4, 8, 24 and 48 h of aphid feeding, and leaves infested by aphids for one week (FIG. 1a). The samples were run on LC-MS under the same conditions as non-targeted metabolite assays. Metabolites of interest were identified by comparing the fragmentation patterns with pure standards, daidzein, prunetin, formononetin, apigenin and kaempferol, obtained from Sigma.

### RNA-seq Library Preparation and Sequencing

Total RNA was extracted from leaves infested by SBA and PA from D3_3, D4_7 and T2_10 which were collected at 0, 4, 8, 24 and 48 h of aphid feeding using TRIzol reagent (Life Technologies), followed by purification using the SV Total RNA isolation kit (Promega). RNA-seq libraries were prepared from the collected tissues described above using a custom high-throughput method for the Illumina RNA-seq library preparation (66). Libraries were prepared from three replicates. These RNA-seq libraries were sequenced at Genomics Sequencing Laboratory (Cornell University) using the HiSeq2500 platform (Illumina), and reads were generated in 150-bp paired-end format.

### De Novo Assembly, Annotation, Differential Expression Analysis and Enrichment Analysis

Preprocessing of raw reads involved Q20-quality trimming (removal of low quality reads with average Phred quality score ≤20 and trimming of low quality bases from the 3′ ends of the reads) and removal of reads containing primer/adaptor sequences and ambiguous reads were done using the SeqPrep and Sickle software. The cleaned reads from each genotype with various treatments were mixed and then de novo assembled using Trinity release v2.1.1 at the parameters of -Trinity -seqType fq -max_memory 50G -output trinity -left left.fastq -right right.fastq -CPU 36 -min_kmer_cov 3 -min_contig_length 350 -bfly_opts “-V 10” (67). Three (D3, D4 and T2) de novo transcriptomes were generated.

To determine the predicted functions, all assembled unigenes were utilized for BLASTx against the following databases with an e-value <1e-5, including NR (NCBI Non-redundant Protein database), Swiss-Prot protein database (Release 2013_03), KEGG (Kyoto Encyclopedia of Genes and Genomes pathway database, Release 63.0), and COG database (Cluster of Orthologous Groups database).

All unigenes from D3, D4 and T2 were pooled to generate a merged assembly. In order to reduce the redundancy of the merged assembly, these three assemblies were first processed by CD-HIT software, with 95% identity, remove redundancy from transcripts derived from homeologous genes or different alleles of the same genes (68).

All of the trimmed reads from individual libraries of each treatment were mapped onto the non-redundant set of transcripts (the merged assembly) to quantify the abundance of transcripts assembled, the calculation of unigene expression used the FPKM method, the aligner Bowtie2 (version 2.2.6) and RSEM method using default parameters, which were able to eliminate the influence of different gene lengths and sequencing levels on the calculation of gene expression (69, 70). Differential expression analysis of the mapped read counts was conducted with edgeR, an estimated absolute value of log 2-fold change of ≥2 and FDR adjusted P-value ≤0.05 were used as the threshold to judge the significance of differential expressed genes (DEGs). GO enrichment analysis was completed using the Python goatools package. KOBAS was used to identify enriched KEGG in the DEGs between controls and aphid treatments (71).

Identification of soybean resistance-related genes was based on the most conserved motif structures of plant resistance proteins, including CC (coiled-coil), KIN (kinase), TIR (Toll-interleukin receptor-like), NBS (nucleotide binding site), and LRR (leucine-rich repeat) finger domains. To identify putative resistant genes in soybeans, all unigenes were used as blastx queries against the reference R-gene PRGdb database (50). Assigning candidate genes to different R gene classes was based on the aforementioned protein domain composition.

qRT-PCR Analysis

The samples from targeted metabolite quantitative analysis were used for RNA isolation and cDNA synthesis (PrimeScript™ RT reagent Kit, TaKaRa, Japan). Gene-specific primers were designed by NCBI Prime-BLAST (Table 1). qRT-PCR (Quantitative reverse transcription polymerase chain reaction) was analyzed using SYBR Green master mix (TaKaRa, Japan) and QuantStudio 6 Flex real-time PCR system (ThermoFisher, USA). The thermal cycling conditions were as follows: 3 min at 94° C., followed by 40 cycles each consisting of 95° C. for 15 s, 60° C. for 30 s, 72° C. for 1 min. Elongation factor 1-alpha was used as an internal control. Each reaction was performed in triplicate and the 2^{-ΔΔct} method was used to calculate the expression levels. Student's t-test was used for statistics analysis.

### Virus Induced Gene Silencing (VIGS) of Candidate Genes Expression and Effects on Aphid Performance

The Bean pod mottle virus vectors pBPMV-IA-V2 and pBPMV-IA-V3 (48) for silencing or transiently expressing genes in soybean were kindly were kindly provided by Steve Whitham, Iowa State University. Previously described protocols for cloning genes into these vectors were followed (48). The primers and the product size for pBPMV2-IFS (CRRK20/CRRK42/LRPK) and pBPMV3-IFS (CRRK20/CRRK42/LRPK) are listed in Table 1. Briefly, for silencing constructs, gene products were cloned into the pBPMV-IA-V2 vector, at the BamHI and XhoI restriction sites. For overexpression constructs, a gene encoding 19 kDa protein of Tomato bushy stunt virus was removed from pBPMV-IA-V3 using the XhoI and SmaI restriction sites, and the gene products of interest were inserted to the same position. BPMV RNA1 and recombinant RNA2 (V2 or V3) clones were mixed and then biolistically bombarded into soybean leaves to initiate systemic infections as well as silencing or overexpression of the target genes. In order to share the same control for silencing and overexpression, empty vectors pBPMV2 and pBPMV3 were co-transformed into control plants. When transforming a silencing vector, the empty vector for overexpression was co-transformed as a control. When transforming an overexpression construct, the empty vector for silencing was con-transformed.

To detect the accumulation of virus, overexpression, and silencing efficiency in the leaves, total RNA was extracted from two weeks after infection. RNA2 was amplified by RT-PCR with RNA2-specific primers to detect virus. Gene expression silencing and overexpression analysis were conducted by qRT-PCR with specific primers for the targeted genes (Table 1). Meanwhile, flavonoids in these plants were measured by LC-MS using the method described above. Finally, three newborn pea aphids or soybean aphids were added to each plant and, after one week, aphid progeny numbers were recorded.

### Statistical Analysis

Aphid fecundity on D3, D4, T2 and their silenced or overexpressed plants was analyzed by ANOVA, followed by a Tukey's honestly significant difference (HSD) post hoc test. For qRT-PCR in time course experiment, three biological replicates were analyzed per treatment, and by ANOVA, followed by Dunnett's post hoc test. For qRT-PCR in the VIGS experiments, five biological replicates were analyzed per treatment, and by ANOVA, followed by a Tukey's HSD post hoc test. All statistical analyses were performed in R.

### Sequences for Use in the Methods Described Herein Below.

CRRK42
>D3_TRINITY_DN45591_c0_g1
(SEQ ID NO: 1)
CTACACTCTTCATGGGTGAGATCTCTGGAGCACTGCACCAAGCCATACCT
CTTCTCAGTGGAACTCAAATTGAAGCCATCCATATAGTACAACTGATTGG
TCTCATTAGTTGCTTTTCTGATCAAACTTCTCATAAAAAATCCTGCCTTT
TCTGAATCTCTGCCATGTTGGAAATGTTTTTGGCTCCAACATCATGCCAT
GTTGAGAATGTTGTAACATTCCCAAAGAAGCTTTCATTAGAGTACCTTAT
GACGCAGAAATCATACCATATGATAGCAGAAACTCTATTGAGGCAGTGCT
GAAGTACTTGTCTGGAAGCAGTAGATACACAGAATTGACAAAAGTATCCA
AACACATCACCACGGCAATCATAGAGGCCATACACAGCACTGTAGTTGTT
TTTTCCTATCGTTTTGTGGTTATAACCTTTGCTTGTGGCTGCATCTGTGG
ATAGCCATGAGAGTATGCTGTTAAGGTTGGTTTGGTATGCATCGCTGAGA
GGTTTTTGGGTGGTGTTATGGCAGTCATCTCCCATGTAGTTTGGTGATTG
TGCCTTTGTATCAAGTGGTTTGATGCTTAGCAATAGCAGGACAGCAAAGG
AATATACTGATTTCACACTGAGTAGTGTTGCATCCATTGAGCACTGTCT
CRRK20
>D4_TRINITY_DN27570_c0_g1
(SEQ ID NO: 2)
AATAAGTACACCACCTGACCACCATTTTGCATAAACACCTTATTTTGCAC
AAACTACAACAGAAATATAAGAAAACACAACCCAGATGGGAAAGAAAAGT
ACAAGATTTGTTTGCTTTACCAACATCAGATCAGCTACTACATTATCCAC
TTTAGAGTTCTGTACCTTTGCCCTCTATAGTGTTCATAAACAAAGCCAAA
CTGAGGAACAAGCATGTAAAGCAAGTCATCAACACGCTGCCAATACAAAA
GCCCCCACCTCTCCAAGAGCATAAGGGGAAGGATAATGATTCCAAAGCCA
AAAATGAATCCCAGCTCCACACTTAGGAAATTCCAGTCAATTGATCCATG
TATTTCATATAGTGTTGGAAGGGAATGTCCCACTCTATCGTTGGTGCAAT
CTTTTAAGGGTGGCCCACATAATCCTTCATTGCCTTCAAAATAACTTGCA
TCAAATGTTTGCATTTGAGCACCTGTTGGGATTTGCCCCCTCAACTGATT
AAATGAGAGATTCAAGTATGCTAGAAAATTTAAACTAGCAAGCTCAAGAG
GGATTTTTCCGCTCAAATTGTTATTTGACATGTCCAAGGATTCAAG

The following examples are provided to illustrate certain embodiments of the invention. They are not intended to limit the invention in any way.

### Example I

Plant defense responses often involve recognition of pest or pathogen attack by R proteins. Most R proteins contain a ligand recognition motif such as a leucine-rich repeat or a signal transduction domain such as the kinase domain (34). Sequences predicted to encode nucleotide binding sites and leucine zippers are shared among many resistance genes (35). Two well-studied examples of R-gene mediated resistance to aphids are the tomato Mi gene (36) and the melon Vat gene (37, 38), which confer resistance against Macrosiphum euphorbiae (potato aphid) and Aphis gossypii (cotton aphid), respectively.

Many specialized metabolites in plants, including glucosinolates, volatile terpenoids, and phenolic compounds, are dedicated to herbivore defense (39). Flavonoids also play an important role in the defense response of plants to insect attack. In several plant species, insect-resistant lines have constitutively more abundant flavonoids or induce them to a higher level in response to herbivory, thereby deterring aphids feeding and inhibiting insect growth (40-43). Therefore, variation in the constitutive or induced flavonoid abundance may account for differences between resistant and susceptible plant varieties.

The gene expression response of G. max to insect herbivory, including by soybean aphids and common cutworms, has been extensively studied using microarrays and RNA-seq. However, only a relatively small number of differentially expressed genes were identified (44-47). In the current study, we combined transcriptomic and metabolomic analyses to investigate the dynamic responses of allotetraploid G. dolichocarpa and its diploid progenitors G. tomentella D3 and G. syndetika to attack by two legume-feeding specialist aphids, A. glycines and A. pisum (FIG. 2A). We show that both species-specific flavonoid accumulation and R genes contribute to aphid resistance. Of particular interest is that allotetraploid G. dolichocarpa has enhanced aphid resistance through the combination of traits from its diploid ancestors.

### Results

Allotetraploid Perennial Soybeans are More Aphid-Resistant than the Diploid Progenitors

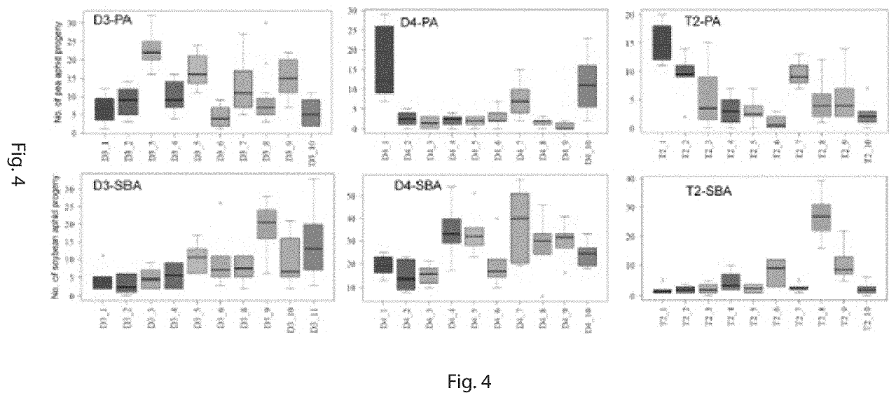

We evaluated the performance of soybean aphids and pea aphids on perennial tetraploid (G. dolichocarpa, T2) and its diploid (G. tomentella D3 and G. syndetika, D4) soybean species using the protocol illustrated in FIG. 1a. For each genotype, ten accessions, which originated from Australia, Papua New Guinea, and Taiwan, were used for bioassays (FIG. 2, Table 1). Soybean aphids produced significantly more nymphs on D4 than on D3 or T2 (FIG. 1b). In contrast, pea aphids produced more nymphs on D3 than on D4 or T2 (FIG. 1d). Although there was a high diversity of aphid performance within each species (FIG. 4), overall T2 combined the aphid resistance traits of the two diploid progenitors. A notable exception was the Taiwanese T2_8 accession, which was much more susceptible to SBA than the nine tested Australian accessions (FIG. 4).

TABLE 1
Perennial Glycine germplasm and locality information for samples
used to investigate aphid resistance.

| CSIRO | | | |
|---|---|---|---|
| Accession | Sample | | |
| Number | Number | Species and ploidy | Locality |
| G1403 | D3_1 | G. tomentella D3 2n = 40 | QLD, Australia |
| G1366 | D3_2 | G. tomentella D3 2n = 40 | QLD, Australia |
| G1380 | D3_3* | G. tomentella D3 2n = 40 | QLD, Australia |
| G1407 | D3_4 | G. tomentella D3 2n = 40 | QLD, Australia |
| G1691 | D3_5 | G. tomentella D3 2n = 40 | QLD, Australia |
| G1746 | D3_6 | G. tomentella D3 2n = 40 | QLD, Australia |
| G1749 | D3_7 | G. tomentella D3 2n = 40 | QLD, Australia |
| G1820 | D3_8 | G. tomentella D3 2n = 40 | QLD, Australia |
| G2586 | D3_9 | G. tomentella D3 2n = 40 | QLD, Australia |
| G1364 | D3_10 | G. tomentella D3 2n = 40 | PNG, Australia |
| G1300 | D4_1 | G. syndetika D4 2n = 40 | QLD, Australia |
| G1410 | D4_2 | G. syndetika D4 2n = 40 | QLD, Australia |
| G1772 | D4_3 | G. syndetika D4 2n = 40 | QLD, Australia |
| G1775 | D4_4 | G. syndetika D4 2n = 40 | QLD, Australia |
| G1777 | D4_5 | G. syndetika D4 2n = 40 | QLD, Australia |
| G1780 | D4_6 | G. syndetika D4 2n = 40 | QLD, Australia |
| G2073 | D4_7* | G. syndetika D4 2n = 40 | QLD, Australia |
| G2321 | D4_8 | G. syndetika D4 2n = 40 | QLD, Australia |
| G2471 | D4_9 | G. syndetika D4 2n = 40 | QLD, Australia |
| G2724 | D4_10 | G. syndetika D4 2n = 40 | QLD, Australia |
| G1134 | T2_1 | G. dolicocarpa T2 2n = 80 | QLD, Australia |
| G1188 | T2_2 | G. dolicocarpa T2 2n = 80 | QLD, Australia |
| G1264 | T2_3 | G. dolicocarpa T2 2n = 80 | QLD, Australia |
| G1286 | T2_4 | G. dolicocarpa T2 2n = 80 | QLD, Australia |
| G1393 | T2_5 | G. dolicocarpa T2 2n = 80 | QLD, Australia |
| G1412 | T2_6 | G. dolicocarpa T2 2n = 80 | QLD, Australia |
| G1769 | T2_7 | G. dolicocarpa T2 2n = 80 | QLD, Australia |
| G1854 | T2_8 | G. dolicocarpa T2 2n = 80 | Taiwan |
| G2320 | T2_9 | G. dolicocarpa T2 2n = 80 | QLD, Australia |
| G2809 | T2_10* | G. dolicocarpa T2 2n = 80 | QLD, Australia |

*Denotes samples used for transcriptomic and metabolomics analyses.

### Transcriptomic and Metabolomic Characterization of Perennial Soybean Infested by Aphids

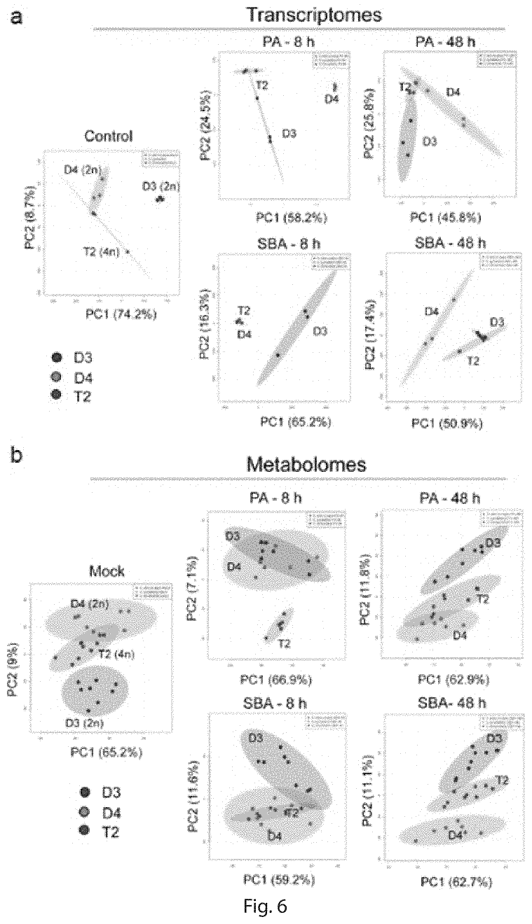

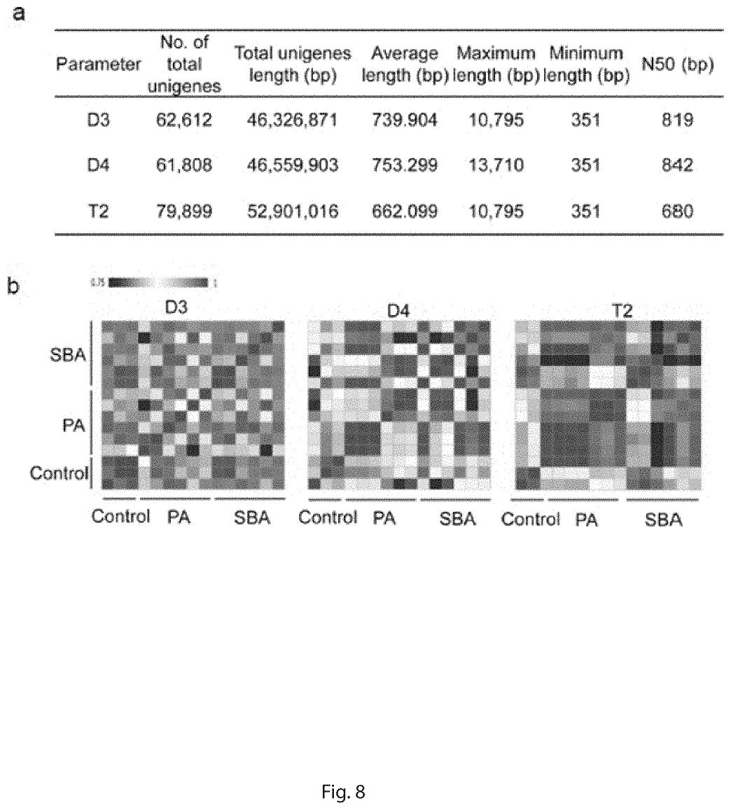

To identify transcriptomic and metabolomic changes of the three perennial soybean species in response to aphid feeding, we chose the accessions with the most divergent resistance phenotypes. As shown in FIG. 4, D3_3 (resistant to SBA and susceptible to PA), D4_7 (resistant to PA and susceptible to SBA), have the greatest difference between PA and SBA progeny production for each of the diploid species. Accession T2_10 (relatively high resistance to both SBA and PA) is representative of the allopolyploid response. cDNA libraries were prepared using three independent RNA samples each from D3_3, D4_7, and T2_10, which were infested by the two aphid species for 0, 8, or 48 h (FIG. 3a). An average of 2.8 million sequence reads were generated for each sample by Illumina sequencing. After removal of low-quality reads, the total number of reads per library ranged from 2 million to 2.5 million. De novo assembly of the RNA-seq reads generated ˜60,000 unique contigs for the diploids D3 and D4, as well as 80,000 for the tetraploid T2, with the lengths ranging from 351 to 13,710 bp and an average length of 710 bp (FIG. 6A). cDNA sequences have been deposited in the NCBI GEO database with the accession number XX. The pairwise correlations of the biological replicates (FIG. 8b) showed a high level of reproducibility (Pearson correlation ≥0.75). With the exception of pea aphids feeding on T2, the number of induced and repressed genes was greater after 48 hours than after 8 hours (FIG. 3b, c). Principal component analysis (PCA) of homologous genes in the three perennial soybean species showed a shift over time (FIG. 6a), with the two SBA-resistant species, D3 and T2 (FIG. 1c), being more similar to each other after 48 h of soybean aphid feeding. The overall gene expression response to PA was different, with the D4 expression response being closer to T2, consistent with the observation that D4 and T2 have higher PA resistance than D2 (FIG. 1b).

HPLC-MS metabolomic analysis of the lines that were used for transcriptomic analysis identified a total of 1791 unique mass features. After 8 hours of feeding by either aphid species, there were more mass features that decreased in abundance than increased in abundance (FIG. 3d). However, this pattern had changed after 48 h of aphid feeding, when there were larger numbers of metabolites with increased abundance. The PCA analysis of the metabolite profiles marginally paralleled that of the transcriptome (FIG. 6b). Notably, after 48 h of aphid feeding the metabolomes of the two PA-resistant species, D4 and T2 (FIG. 1b), were more similar to one another than to D3. Similarly, at 48 h the metabolomes of the SBA-resistant species, D3 and T2 (FIG. 1c), were more similar to one another than to D4.

To investigate the biological functions of genes that were differentially regulated by aphid infestation, we mapped these genes to terms in the KEGG database to identify significantly enriched metabolic and signal transduction pathways. Among the mapped pathways, thirteen were significantly enriched (FDR≤0.05) after 8 h of aphid infestation (FIG. 3f). Notably, specific enrichment was observed in phenylpropanoid biosynthesis, flavonoid biosynthesis, and linoleic acid metabolism (FIG. 3f). After 48 h of aphid infestation, the metabolic pathways identified at 8 h were still significantly enriched in most aphid species—time point combinations. Additionally, some pathways related to both primary (starch and sucrose metabolism) and secondary (terpenoid biosynthesis) metabolism were significantly enriched (FIG. 3g).

Flavones and Isoflavones are Associated with Resistance Against Different Aphid Species.

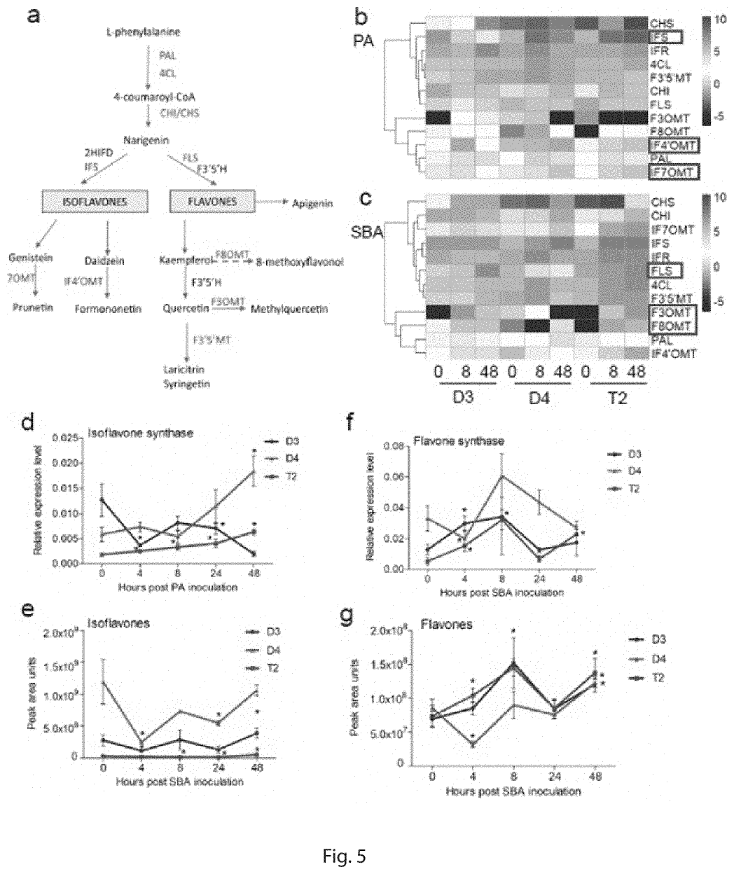

Biosynthetic pathways for flavonoids (FIG. 5a), a class of specialized metabolites that are involved in plant defense, were significantly regulated in response to aphid infestation (FIG. 5b, c). Marked differences in flavonoid gene expression profiles were observed for resistant and susceptible perennial soybean lines after PA and SBA infestation. The expression levels of genes involved in the isoflavone biosynthesis pathway (isoflavone synthase, isoflavone 4′-O-methyltransferase, and isoflavone-7-O-methyltransferase) were higher in PA-resistant plants (D4 and T2) than susceptible plants (D3) after 48 h of PA feeding (FIG. 5b). In contrast, the expression levels of genes involved in the flavone biosynthesis pathway (flavonoid 8-O-methyltransferase, flavonoid 3′,5′-methyltransferase, and flavonol 3-O-methyltransferase) were higher in SBA resistant plants (D3 and T2) than susceptible plants (D4) after 48 of SBA feeding (FIG. 5c).

Given this pattern, we examined the isoflavone responses to PA and the flavone responses to SBA in more detail at 0, 4, 8, 24, and 48 hours after the initiation of aphid feeding. Expression levels of isoflavone synthase and flavone synthase were measured as indicators of the relative contributions of the two branches of the flavonoid biosynthetic pathway in the perennial soybean response to aphid feeding. The sum of three identified isoflavones, (daidzein, prunetin, and fomononetin) and the sum of two identified flavones (kaempferol and apigenin) were used to estimate the relative abundance of isoflavones and flavones, respectively. In response to feeding by PA, the expression pattern of isoflavone synthase increased in the resistant species D4 and T2, decreased in the sensitive D3 species, the expression pattern for isoflavone synthase seems more similar between T2 and D4 (susceptible) than between T2 and D3 (resistant), in both T2 and D4, IFS is induced by aphids in both T2 and D4, reaching its highest level at 48 h, whereas in D3 the expression level was similar with the constitutive level at all time points. (FIG. 5d). Consistent with the gene expression patterns, isoflavones were more abundant in D4 and T2 than in D3 after 48 h of PA feeding (FIG. 5e). In response to SBA feeding, both flavone synthase gene expression level (FIG. 5f) and flavone abundance (FIG. 5g) increased at earlier time points in the resistant D3 and T2 species than in the sensitive D4 species.

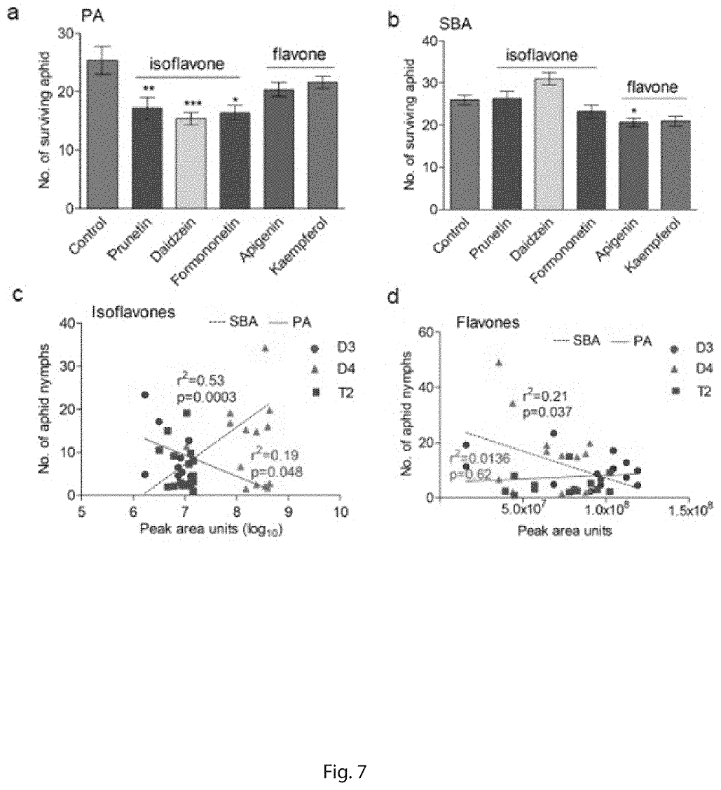

To determine whether flavonoids deter aphid feeding, we added isoflavones (daidzein, prunetin, and formononetin) and flavones (apigenin and kaempferol) to the aphid artificial diet at concentrations similar with those found in perennial soybean leaves separately, and recorded the survival rate of aphids after 2 days. There was higher mortality of PA after feeding by diet with isoflavones (daidzein, prunetin, and formononetin) compared with feeding by control diet or with flavones (apigenin and kaempferol) (FIG. 7a). When fed by diet with flavone apigenin, the mortality of SBA increased, while no effect of isoflavones on SBA performance (FIG. 7a).

To confirm the effect of flavonoid abundance on aphid performance, we extended our analysis to seven isolates of each soybean species. Isoflavones and flavones were measured in leaves of soybean plants that had been infested for 7 days in an experiment such as that illustrated in FIG. 1a. Whereas PA performance was negatively correlated with isoflavone abundance (r²=0.1906, p=0.048), SBA performance was positively correlated (r²=0.53, p=0.0003; FIG. 7c). Conversely, flavone abundance was negatively correlated with SBA performance (r²=0.206, p=0.037) and showed no significant correlation with PA performance (r²=0.0136, p=0.62; FIG. 7d).

### Manipulation of Flavonoid Biosynthetic Genes Influences Aphid Resistance

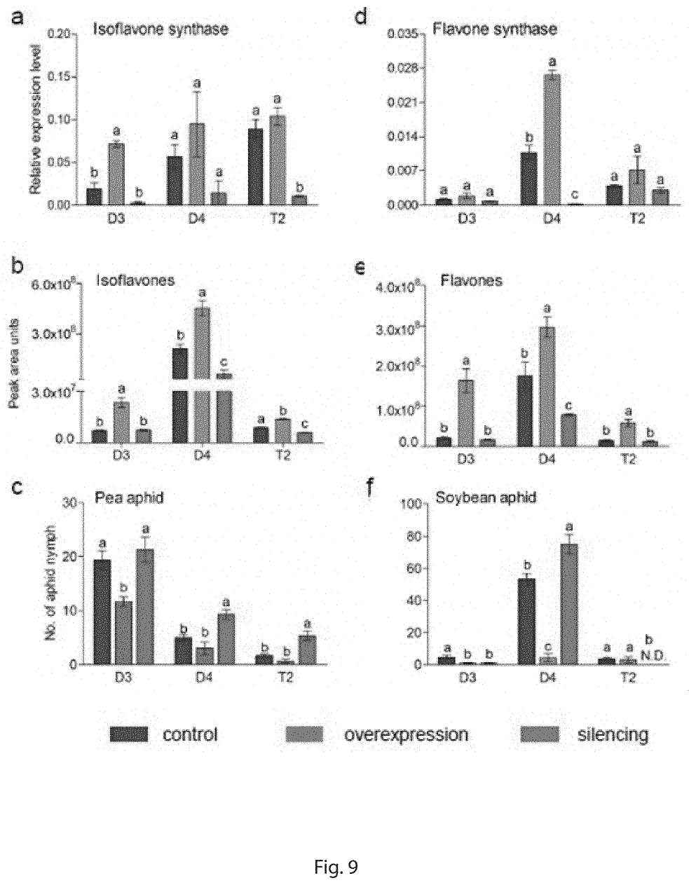

To investigate the relative importance of isoflavone synthase and flavone synthase in aphid resistance, we made virus induced gene silencing (VIGS) and overexpression constructs based on a Bean pod mottle virus vector (BPMV) (48). Two weeks after BPMV infection of D3_3, D4_7, and T2_10, with the overexpression construct, isoflavone synthase gene expression levels were consistently increased but not statistically significant, as measured by quantitative RT-PCR (FIG. 9a). Nevertheless, isoflavone levels were increased in these lines and PA reproduction was decreased (FIG. 9b, FIG. 9c). Conversely, VIGS decreased isoflavone synthesis expression, thereby decreasing isoflavone abundance and increasing PA reproduction (FIG. 9a-c). A flavone synthase overexpression construct also did not consistently result in significantly increased gene expression levels (FIG. 9d), but increased flavone abundance (FIG. 9e) and decreased SBA progeny production (FIG. 9f) in D3 and D4. The flavone VIGS construct caused a significant reduction in gene expression only in D4, which was associated with decreased flavone abundance and increased SBA reproduction (FIG. 9d-f).

### Putative Receptor-Kinase Genes Confer Aphid Resistance in Perennial Soybean

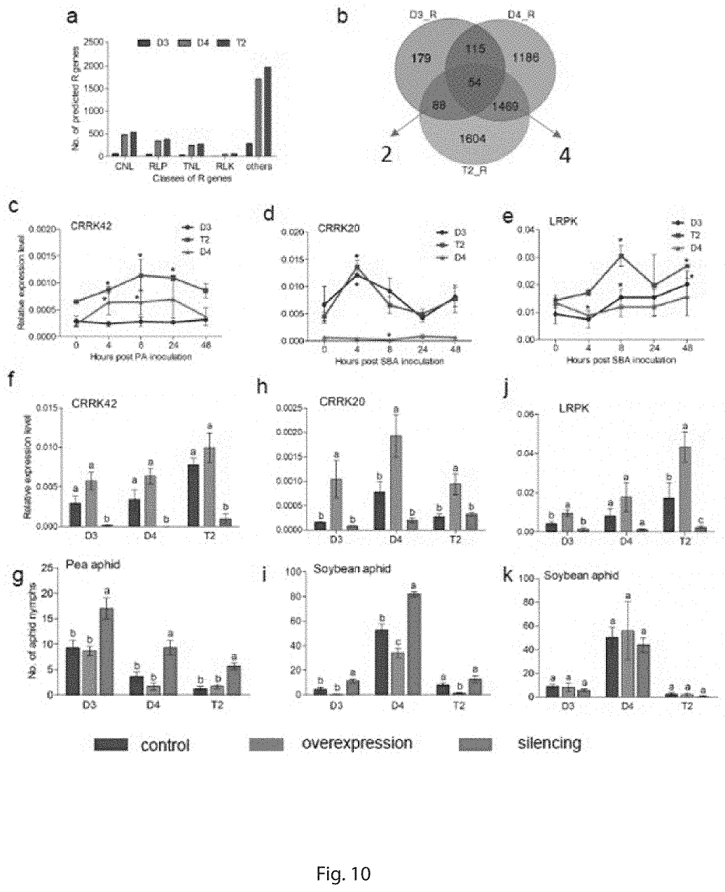

R genes have been associated with aphid resistance in several plant species (49). We therefore used BLAST searches of the Plant Resistance Gene database (PRGdb) (50) to identify different classes of predicted R genes (FIG. 10a). There were a total of 436 predicted R genes in the assembled unigenes of the D3_3 transcriptome, 2,824 in the D4_7 transcriptome, and 3,215 in the T2_10 transcriptome, these predicted R gene were classified into 5 classes based on the presence of specific domains (FIG. 10a, FIG. 10b). Of these, 1,469 predicted R genes were present in D4 and T2 but not in the D3 unigene set, which might indicate involvement in resistance to PA. In contrast, SBA resistance may be associated with one or more of 88 predicted R genes that were present in D3 and T2 but not in the D4 assembled unigenes.

To narrow down the list of candidate R genes, we did additional BLAST comparisons to Illumina sequencing reads that had not been assembled into the unigene set. This showed that, among 88 unigenes identified as unique to D3 and T2, only two had no sequence identity to the D4 unassembled Illumina reads, a predicted cysteine-rich receptor-like protein kinase 20 (CRK20) and a predicted G-type lectin S-receptor-like serine/threonine-protein kinase (LRK) which belong to the RLP class. Similarly, among 1,486 unigenes identified as unique to D4 and T2, only 4 had no sequence identity to the D3 Illumina unassembled reads. These included a predicted cysteine-rich receptor-like protein kinase 42 (CRK42) (RLP class), a predicted phytosulfokine LRR receptor kinase 1 (RLP class), a predicted serine/threonine-protein kinase (RLP class), and a predicted CBL-interacting protein kinase 1 (RLK class). Higher expression levels in resistant than in susceptible perennial soybean species were used as a further indicator of potential involvement in aphid resistance. This expression analysis narrowed the list of candidate R gene to CRK42 for PA resistance, and CRRK20 and LRPK for SBA resistance (FIG. 10c-e).

We next determined whether these predicted R genes are involved in aphid resistance by silencing or overexpressing them using the BPMV vector. In the case of CRK42, there was consistently significant silencing and the trend of overexpression but not significant (FIG. 10f). In congruence with the gene expression changes, PA produced more offspring on the CRK42-silenced plants, but were not significantly affected on the overexpression plants (FIG. 10g). CRK20 was overexpressed, but not significantly silenced in the three soybean species (FIG. 10h). In this case, SBA produced fewer progeny on the overexpression lines than on the controls, but aphid reproduction was only increased on D3 and D4. LRK expression was significantly affected by overexpression or silencing only in T2. However, no significant effect was detected on SBA performance.

### Discussion

Polyploid plants have often been observed to have greater fitness, as evident by their wider distribution and enhanced adaptability (2,3). These evolutionary advantages have been attributed in part to their enhanced insect herbivore resistance based on observational studies (5-7). Previous research showed that allotetraploid Nicotiana species are more resistant to Manduca sexta (tobacco hornworm) attack and allotetraploid G. tomentella are more resistant leaf rust infection than their diploid progenitors (10, 51). Consistent with these previous studies, we show that allopolyploid G. dolichocarpa has combined the resistance against two different aphid species that occur separately in the two diploid progenitors, G. tomentella D3 and G. syndetika (FIG. 1b). Using transcriptomic and metabolomic methods, we identified regulation of flavonoid metabolism and stacking of R genes from both parental species as likely physiological and molecular mechanisms underlying the higher aphid resistance in the allotetraploid G. dolichocarpa (FIG. 2 & FIG. 6). We further demonstrated the relevance of flavonoid metabolic genes and predicted R genes by showing that the manipulation in their expression can result in significant changes in insect herbivore resistance (FIG. 9 & FIG. 10). Hence, this study provides experimental evidence that allopolyploid plants have an expanded range of insect resistance through a combination of defenses from diploid progenitors.

Flavonoids, the most abundant specialized metabolites in soybeans, are known to contribute to wide arrays of biotic interactions (52). In diverse plant species flavonoids have shown to have cytotoxic, feeding deterrent, and growth-inhibitory effects against insect herbivores (41). Rutin and genistin, which are constitutively produced in soybean PI 227697 leaves, negatively affect the performance of Trichoplusia ni and Anticarsia gemmatalis larvae (53). While the bioactivity and structural diversity of flavonoids are well documented and their biosynthetic pathways have been largely elucidated, there have been few studies comparing the biological effects of different classes of flavonoids. In the current study, we found that two classes of flavonoids, namely flavones and isoflavones, are associated with resistance against different aphid species (FIG. 5b & FIG. 5c). This hypothesis, which is derived from the differential expression patterns of flavone and isoflavone synthases, was further supported by in vitro insect feeding assays results, and insect performance assays on genetically manipulated plants (FIG. 7 & FIG. 9). Previously, it has been reported that the same flavonoid compounds can serve as either deterrent or attractant to different insect species (54, 55). Our results further highlight that the chemical ecological outcome of plant-insect interactions is a function of both the chemical structures of phytochemicals, as well as the identity of the perceiving insects. Intriguingly, the constitutive level of flavonoids is higher in the two diploids, D3 and D4, compared with T2, this extra metabolic flux might be compensated by lower growth rate of D3 and D4. When attacked by aphids, the level of both flavone and isoflavones in the allotetraploid G. dolichocarpa species increased more rapidly than its diploids, implies a greater metabolic flux and energy investment to the flavonoid biosynthesis pathway.

In addition to innate chemical defense, plant resistance to insect herbivores is also known to be regulated by resistance, or R proteins (56). These proteins usually comprise a ligand recognition domain (e.g. leucine rich repeat, or coiled-coil) and signaling kinase domain (57). Unlike chemical defenses, which tend to be broad spectrum and constitutive, R-gene-mediated resistance is only turned on in presence of specific ligands. Compared to the biochemical defense mechanisms, R genes provide more efficient resistance to the plants, but also present a much stronger selective pressure on the insects (58). Therefore, in agricultural settings, though R genes are highly protective of the crops upon initial introduction, their efficacy exponentially decays as the pest populations rapidly develop resistance (59). As a solution to this problem, gene pyramiding, a technique that brings together multiple genetic sources of resistance through conventional crossing, has been proposed (60). In this study, we observed evidence of R gene pyramiding in natural allotetraploid in nature, which combined R genes encoded in the two diploid parental genomes. The importance of these putative R genes is confirmed by the increased aphid susceptibility in the overexpressing and expression-silenced plants (FIG. 10h&i).

Allopolyploidy is a genetic process that includes both whole genome duplication and hybridization. In our study system, we cannot parse out the effects of these two components. However, the nature of the biochemical and molecular phenotypes that we observed could shed light on their genetic basis. For flavonoid abundance, the allotetraploid G. dolichocarpa species appears to have an intermediate state between the two diploid parents, both constitutively and after aphid induction. This pattern is consistent with the expected outcome of a hybridization event, where two active enzymes are brought together to compete for the same chemical substrate. Whole genome duplication per se, on the other hand, would have reinforced any bias in metabolic flux existing in the diploid parents. The CRK20 gene expression in the allotetraploid is most likely due to the presence of the D3 genome from G. tomentella, since expression is almost undetectable in the D4 genome. This would be another example of hybrid vigor expected even from a diploid hybrid. The CRK42 gene expression, however, does show an additive pattern, such that its constitutive expression level in the allotetraploids is approximately twice as high as in either diploid parent. This would suggest that a whole genome duplication event per se might be sufficient to result in higher CRK42 expression. Interestingly, CRK42 expression is only inducible in G. syndetika (D4) and G. dolichocarpa (T2), which would suggest the aphid-responsive regulatory element could be inherited through hybridization per se.

As is the case for many crop species, G. max evolution under domestication has led to reduced genetic diversity, which has a negative effect on the ability to adapt to different environments (61). Conversely, the wild relatives of soybeans and other crops tend to be more tolerant of changes in their environments due to their greater genetic diversity. These adaptive traits could be of agricultural relevance, as some resistance to specific diseases and tolerance to abiotic extremes can to be re-introduced into domesticated crops through breeding (62). Quantitative trait loci conferring resistance to several soybean aphid biotypes have been reported; eight individual Rag (Resistance to Aphis glycines) genes have been mapped, and been numbered from Rag1 through Rag5, with three provisional genes (26). However, these Rag genes have been overcome by new aphid biotypes (25). Thus, it should be possible to introduce the predicted R genes that we have identified in wild perennial soybeans into G. max to develop more resistant cultivars.

In this study, we have addressed the ecological significance of polyploidy. Our results show that allotetraploid soybeans are more resistant to aphids than their diploid progenitors. We further confirm that different classes of flavonoids confer resistance to different types of aphids. In addition, we identified two predicted R genes conferring resistance to soybean aphid or pea aphid, which might provide insights for breeding aphid-resistant soybean cultivars.

### REFERENCES

1. Soltis D E V C, Marchant D B, Soltis P S. (2016) Polyploidy: Pitfalls and paths to a paradigm. American Journal of Botany 103(7):1146-1166.
2. Van de Peer Y, Mizrachi E, & Marchal K (2017) The evolutionary significance of polyploidy. Nat Rev Genet 18(7):411-424.
3. Levin D A (1983) Polyploidy and novelty in flowering plants. The American Naturalist 122(1):1-25.
4. to Beest M, et al. (2011) The more the better? The role of polyploidy in facilitating plant invasions. Annals of Botany:mcr277.
5. Ramsey J & Ramsey T S (2014) Ecological studies of polyploidy in the 100 years following its discovery. Philos Trans R Soc Lond B Biol Sci 369(1648).
6. Harper J L, Clatworthy J, McNaughton I, & Sagar G (1961) The evolution and ecology of closely related species living in the same area. Evolution 15(2):209-227.
7. Thompson J N, Cunningham B M, Segraves K A, Althoff D M, & Wagner D (1997) Plant polyploidy and insect/plant interactions. The American Naturalist 150(6):730-743.
8. Segraves K & Thompson J (1999) Plant polyploidy and pollination: floral traits and insect visits to diploid and tetraploid Heuchera grossulariifolia. Evolution: 1114-1127.
9. Halverson K, Heard S B, Nason J D, & Stireman J O (2008) Differential attack on diploid, tetraploid, and hexaploid Solidago altissima L. by five insect gallmakers. Oecologia 154(4):755-761.
10. Lou Y & Baldwin I T (2003) Manduca sexta recognition and resistance among allopolyploid Nicotiana host plants. Proceedings of the National Academy of Sciences 100(suppl 2): 14581-14586.
11. Schoen D, Burdon J, & Brown A (1992) Resistance of Glycine tomentella to soybean leaf rust Phakopsora pachyrhizi in relation to ploidy level and geographic distribution. TAG Theoretical and Applied Genetics 83(6):827-832.
12. Soltis D E, Buggs RJA, Doyle J J, & Soltis P S (2010) What we still don't know about polyploidy. Taxon 59(5):1387-1403.
13. Schlueter J A, et al. (2004) Mining EST databases to resolve evolutionary events in major crop species. Genome 47(5):868-876.
14. Doyle J J, Doyle J L, Rauscher J T, & Brown A (2004) Evolution of the perennial soybean polyploid complex (Glycine subgenus Glycine): a study of contrasts. Biological Journal of the Linnean Society 82(4):583-597.
15. Bombarely A, Coate J E, & Doyle J J (2014) Mining transcriptomic data to study the origins and evolution of a plant allopolyploid complex. PeerJ 2:e391.
16. Doyle J J, Doyle J L, Rauscher J T, & Brown A (2004) Diploid and polyploid reticulate evolution throughout the history of the perennial soybeans (Glycine subgenus Glycine). New Phytologist 161(1):121-132.
17. Sherman-Broyles S B A, Powell A F, Doyle J L, Egan A N, Coate J E, Doyle J J (2014) The Wild Side of a Major Crop: Soybean's Perennial Cousins from Down Under. American Journal of Botany 101(10):1651-1665.
18. Coate J E, et al. (2012) Anatomical, biochemical, and photosynthetic responses to recent allopolyploidy in Glycine dolichocarpa (Fabaceae). American Journal of Botany 99(1):55-67.
19. Powell A F & Doyle J J (2016) Enhanced rhizobial symbiotic capacity in an allopolyploid species of Glycine (Leguminosae). American journal of botany 103(10):1771-1782.
20. Coate J E P A, Owens T G, Doyle J J (2013) Transgressive physiological and transcriptomic responses to light stress in allopolyploid Glycine dolichocarpa (Leguminosae). Heredity (Edinb) 110(2): 160-170.
21. Ilut D C C J, Luciano A K, Owens T G, May G D, Farmer A, Doyle J J (2012) A comparative transcriptomic study of an allotetraploid and its diploid progenitors illustrates the unique advantages and challenges of RNA-seq in plant species. American Journal of Botany 99(2):383-396.
22. Ragsdale D W, Voegtlin D J, & O'neil R J (2004) Soybean aphid biology in North America. Ann Entomol Soc Am 97(2):204-208.
23. Hill C B, Li Y, & Hartman G L (2004) Resistance to the soybean aphid in soybean germplasm. Crop science 44(1):98-106.
24. Hill C, Chirumamilla A, & Hartman G (2012) Resistance and virulence in the soybean-Aphis glycines interaction. Euphytica 186(3):635-646.
25. Kim K S, Hill C B, Hartman G L, Mian M, & Diers B W (2008) Discovery of soybean aphid biotypes. Crop Science 48(3):923-928.
26. Hesler L S, et al. (2013) Performance and prospects of Rag genes for management of soybean aphid. Entomologia Experimentalis et Applicata 147(3):201-216.
27. Ferrari J, Via S, & Godfray HCJ (2008) Population differentiation and genetic variation in performance on eight hosts in the pea aphid complex. Evolution 62(10):2508-2524.
28. Via S, Bouck A C, & Skillman S (2000) Reproductive isolation between divergent races of pea aphids on two hosts. II. Selection against migrants and hybrids in the parental environments. Evolution 54(5):1626-1637.
29. Peccoud J, Ollivier A, Plantegenest M, & Simon J C (2009) A continuum of genetic divergence from sympatric host races to species in the pea aphid complex. Proceedings of the National Academy of Sciences 106(18):7495-7500.
30. Guo S, Kamphuis L G, Gao L, Edwards O R, & Singh K B (2009) Two independent resistance genes in the Medicago truncatula cultivar Jester confer resistance to two different aphid species of the genus Acyrthosiphon. Plant signaling & behavior 4(4):328-331.
31. Gao L-L, Klingler J P, Anderson J P, Edwards O R, & Singh K B (2008) Characterization of pea aphid resistance in Medicago truncatula. Plant Physiology 146(3):996-1009.
32. Julier B, Bournoville R, Landré B, Ecalle C, & Carré S (2004) Genetic analysis of lucerne (Medicago sativa L.) seedling resistance to pea aphid (Acyrthosiphon pisum Harris). Euphytica 138(2):133-139.
33. Klingler J P, Nair R M, Edwards O R, & Singh K B (2009) A single gene, AIN, in Medicago truncatula mediates a hypersensitive response to both bluegreen aphid and pea aphid, but confers resistance only to bluegreen aphid. Journal of experimental botany:erp244.
34. Hammond-Kosack K E & Jones J D (1997) Plant disease resistance genes. Annual review of plant biology 48(1):575-607.
35. Staskawicz B J, Ausubel F M, Baker B J, Ellis J G, & Jones J D (1995) Molecular genetics of plant disease resistance. Science 268(5211):661.
36. Rossi M, et al. (1998) The nematode resistance gene Mi of tomato confers resistance against the potato aphid. Proceedings of the National Academy of Sciences 95(17):9750-9754.
37. Boissot N, et al. (2010) Mapping and validation of QTLs for resistance to aphids and whiteflies in melon. Theoretical and Applied Genetics 121(1):9-20.
38. Pauquet J, et al. (2004) Map-based cloning of the Vat gene from melon conferring resistance to both aphid colonization and aphid transmission of several viruses. Proceedings of Cucurbitaceae, pp 325-329.
39. Mithofer A & Boland W (2012) Plant defense against herbivores: chemical aspects. Annual review of plant biology 63:431-450.
40. Dreyer D L & Jones K C (1981) Feeding deterrency of flavonoids and related phenolics towards Schizaphis graminum and Myzus persicae: aphid feeding deterrents in wheat. Phytochemistry 20(11):2489-2493.
41. Simmonds M S (2003) Flavonoid-insect interactions: recent advances in our knowledge. Phytochemistry 64(1):21-30.
42. Lattanzio V, Arpaia S, Cardinali A, Di Venere D, & Linsalata V (2000) Role of endogenous flavonoids in resistance mechanism of Vigna to aphids. Journal of Agricultural and Food Chemistry 48(11):5316-5320.
43. Golawska S & Lukasik I (2012) Antifeedant activity of luteolin and genistein against the pea aphid, Acyrthosiphon pisum. Journal of pest science 85(4):443-450.
44. Li Y, et al. (2008) Soybean defense responses to the soybean aphid. New Phytologist 179(1):185-195.
45. Wang Y, Wang H, Fan R, Yang Q, & Yu D (2014) Transcriptome analysis of soybean lines reveals transcript diversity and genes involved in the response to common cutworm (Spodoptera litura Fabricius) feeding. Plant, cell & environment 37(9):2086-2101.
46. Studham M E & MacIntosh G C (2013) Multiple phytohormone signals control the transcriptional response to soybean aphid infestation in susceptible and resistant soybean plants. Molecular Plant-Microbe Interactions 26(1):116-129.
47. Prochaska T J, et al. (2015) Transcriptional responses of tolerant and susceptible soybeans to soybean aphid (Aphis glycines). Arthropod-Plant Interactions 9(4):347-359.
48. Whitham S A, et al. (2016) Virus-induced gene silencing and transient gene expression in soybean (Glycine max) using Bean Pod Mottle Virus infectious clones. Current Protocols in Plant Biology: 263-283.
49. Dogimont C, Bendahmane A, Chovelon V, & Boissot N (2010) Host plant resistance to aphids in cultivated crops: genetic and molecular bases, and interactions with aphid populations. Comptes rendus biologies 333(6):566-573.
50. Sanseverino W, et al. (2009) PRGdb: a bioinformatics platform for plant resistance gene analysis. Nucleic acids research 38(suppl 1):D814-D821.
51. Schoen D, Burdon J, & Brown A (1992) Resistance of Glycine tomentella to soybean leaf rust Phakopsora pachyrhizi in relation to ploidy level and geographic distribution. Theoretical and Applied Genetics 83(6):827-832.
52. Mierziak J, Kostyn K, & Kulma A (2014) Flavonoids as important molecules of plant interactions with the environment. Molecules 19(10):16240-16265.
53. Hoffmann-Campo C B, Ramos Neto J A, Oliveira MCNd, & Oliveira L J (2006) Detrimental effect of rutin on Anticarsia gemmatalis. Pesquisa Agropecuária Brasileira 41:1453-1459.
54. Tabashnik B E (1987) Plant secondary compounds as oviposition deterrents for cabbage butterfly, Pieris rapae (Lepidoptera: Pieridae). Journal of chemical ecology 13(2):309-316.
55. War A R, et al. (2012) Mechanisms of plant defense against insect herbivores. Plant signaling & behavior 7(10):1306-1320.
56. Ellis J, Dodds P, & Pryor T (2000) The generation of plant disease resistance gene specificities. Trends in plant science 5(9):373-379.
57. Bent A F (1996) Plant disease resistance genes: function meets structure. The Plant Cell 8(10):1757.
58. Pink D & Puddephat I (1999) Deployment of disease resistance genes by plant transformation—a ‘mix and match’ approach. Trends in plant science 4(2):71-75.
59. Meeusen R L & Warren G (1989) Insect control with genetically engineered crops. Annu Rev Entomol 34(1):373-381.
60. Haverkort A, et al. (2008) Societal costs of late blight in potato and prospects of durable resistance through cisgenic modification. Potato research 51(1):47-57.
61. Esquinas-Alcázar J (2005) Protecting crop genetic diversity for food security: political, ethical and technical challenges. Nature Reviews Genetics 6(12):946-953.
62. Warschefsky E, Penmetsa R V, Cook D R, & von Wettberg E J (2014) Back to the wilds: tapping evolutionary adaptations for resilient crops through systematic hybridization with crop wild relatives. American journal of botany 101(10):1791-1800.
63. Rauscher J T, Doyle J J, & Brown A (2004) Multiple origins and nrDNA internal transcribed spacer homeologue evolution in the Glycine tomentella (Leguminosae) allopolyploid complex. Genetics 166(2):987-998.
64. Russell C W, et al. (2014) Matching the supply of bacterial nutrients to the nutritional demand of the animal host. Proceedings of the Royal Society of London B: Biological Sciences 281(1791):20141163.
65. Wille B & Hartman G (2008) Evaluation of artificial diets for rearing Aphis glycines (Hemiptera: Aphididae). Journal of economic entomology 101(4):1228-1232.
66. Zhong S, et al. (2011) High-throughput illumina strand-specific RNA sequencing library preparation. Cold Spring Harbor Protocols 2011(8):pdb. prot5652.
67. Grabherr M G, et al. (2011) Full-length transcriptome assembly from RNA-Seq data without a reference genome. Nature biotechnology 29(7):644-652.
68. Li W & Godzik A (2006) Cd-hit: a fast program for clustering and comparing large sets of protein or nucleotide sequences. Bioinformatics 22(13):1658-1659.
69. Trapnell C, et al. (2010) Transcript assembly and quantification by RNA-Seq reveals unannotated transcripts and isoform switching during cell differentiation. Nature biotechnology 28(5):511-515.
70. Li B & Dewey C N (2011) RSEM: accurate transcript quantification from RNA-Seq data with or without a reference genome. BMC bioinformatics 12(1):323.
71. Wang Z, et al. (2011) Characterization and development of EST-derived SSR markers in cultivated sweetpotato (Ipomoea batatas). BMC plant biology 11(1):139.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated polynucleotide selected from the group consisting of: a) a polynucleotide encoding a polypeptide involved in resistance to aphids of SEQ ID NO: 1 or a polynucleotide having at least 95% identity to SEQ ID NO: 1; said polynucleotide optionally comprising a promoter sequence operably linked to SEQ ID NO:1 or said polynucleotide having at least 95% identity to SEQ ID NO: 1, said promoter being operable in a plant.

2. An isolated polynucleotide selected from the group consisting of: a) a polynucleotide encoding a polypeptide involved in resistance to aphids of SEQ ID NO: 2 or a polynucleotide having at least 95% identity to SEQ ID NO: 2; said polynucleotide optionally further comprising a promoter sequence operably linked to SEQ ID NO:2 or said polynucleotide having at least 95% identity to SEQ ID NO: 2, said promoter being operable in a plant.

3. An expression cassette comprising the polynucleotide as claimed in claim 1 and/or claim 2, further comprising a nucleic acid encoding a selectable marker.

4. A recombinant vector comprising the polynucleotide as claimed in claim 1 and/or claim 2.

5. The recombination vector of claim 4, wherein said vector is selected from the group consisting of a plasmid, a viral vector, and an agrobacteria vector.

6. A transgenic Glycine max plant cell comprising the polynucleotide as claimed in claim 1 and/or claim 2 operably linked to a promoter, said promoter being operable in said plant cell.

7. A method for producing a transgenic soy bean plant resistant to aphid infestation comprising contacting a Glycine max plant cell with the vector of claim 4, and regenerating a transgenic plant therefrom.

8. The plant cell of claim 6, which is present in a transgenic Glycine max plant.

9. A recombinant vector comprising the expression cassette as claimed in claim 3.

10. The recombinant vector of claim 3 which is a plant viral vector.

11. A method for modulating expression of SEQ ID NOS 1 and or 2 in order to identify agents which alter plant defense response to aphid infestation in a soy bean plant, comprising,
a) contacting said plant cell with an expression vector which encodes i) one or more siRNAs which down modulate SEQ ID NOS: 1 and 2 expression levels; or
ii) a nucleic acid encoding SEQ ID NO: 1 and or SEQ ID NO: 2; thereby transforming said plant cell; and regenerating a plant from said cell and altering resistance to aphid infestation therein;
b) contacting the leaves from the plants of a) with an aphid population in the presence and absence of a test agent, and;
c) identifying agents which alter aphid feeding levels in the presence of said agent and altering aphid infestation in soy plants.
